(19) United States
(12) Patent Application Publication
(10) Pub. No.: US 2006/0140912 A9
(43) Pub. Date: Jun. 29, 2006

(54) Methods for enhancing engraftment of purified hematopoietic stem cells in allogeneic recipients

(76) Inventor: Suzanne T. Ildstad, Prospect, KY (US)

Correspondence Address: FISH & RICHARDSON P.C., PO BOX 1022, MINNEAPOLIS, MN, 55440-1022, US

(21) Appl. No.: 10/438,259

(22) Filed: May 14, 2003

Related U.S. Application Data
(63) Continuation of application No. PCT/US01/45303, filed on Nov. 14, 2001.
(60) Provisional application No. 60/248,889, filed on Nov. 14, 2000.

(51) Int. Cl. A61K 45/00; A61N 5/00; A61K 31/66; C12N 5/08

(52) U.S. Cl. 424/93.7; 435/372; 600/1; 514/109

(57) ABSTRACT

This invention provides a method of achieving a higher rate of allogeneic hematopoietic stem cell engraftment by either (i) matching the major histocompatibility complex class I K locus between donors and recipients or (ii) identifying how class I K on HSC interact with FC (CD8/33Kd receptor complex) works thus allowing one to bypass the need for FC. The MHC loci which are essential for curable engraftment of purified allogeneic HSC are identified by the methods of this invention. This invention further demonstrates that the MHC class I K molecule is essential for maintaining the self-renewal capability of purified HSC. Moreover, interaction between the HSC and FC via the MHC class I K molecule provides a regulatory function to promote engraftment and survival of allogeneic HSC.

### CROSS-REFERENCE TO RELATED APPLICATIONS

This application is a Section 371 filing of PCT/US01/45303, filed Nov. 14, 2001, which claims priority to U.S. Provisional Application Serial No. 60/248,889, filed Nov. 14, 2000, the disclosures of which are incorporated herein by reference.

### CONTRACTUAL ORIGIN OF THE INVENTION

This research was supported in part by the National Institutes of Health grant R01 DK 52294 (S.T.). The U.S. government has certain rights in the invention.

### BACKGROUND OF THE INVENTION

1. Field of the Invention

The present invention relates to a specific major histocompatibility complex (MHC) molecule that strongly influences engraftment of hematopoietic stem cells (HSC) mediated by facilitating cells and more particularly that this MHC molecule is essential for maintaining the self-renewal capability of purified HSC.

2. Description of the State of Art

The transfer of living cells, tissues, or organs from a donor to a recipient, with the intention of maintaining the functional integrity of the transplanted material in the recipient defines transplantation. Transplants are categorized by site and genetic relationship between the donor and recipient. An autograft is the transfer of one's own tissue from one location to another; a syngeneic graft (isograft) is a graft between identical twins; an allogeneic graft (homograft) is a graft between genetically dissimilar members of the same species; and a xenogeneic graft (heterograft) is a transplant between members of different species.

A major goal in solid organ transplantation is the permanent engraftment of the donor organ without a graft rejection immune response generated by the recipient, while preserving the immunocompetence of the recipient to respond to other foreign antigens. Typically, in order to prevent host rejection responses, nonspecific immunosuppressive agents such as cyclosporine, methotrexate, steroids and FK506 are used. These agents must be administered on a daily basis and if stopped, graft rejection usually results. However, a major problem in using nonspecific immunosuppressive agents is that they function by suppressing all aspects of the immune response, thereby greatly increasing a recipient's susceptibility to infections and other diseases, including cancer.

Furthermore, despite the use of immunosuppressive agents, chronic graft rejection still remains a major source of morbidity and mortality in human organ transplantation. Most human transplants fail within 10 years without permanent graft acceptance. Only 50% of heart transplants survive 5 years and 20% of kidney transplants survive 10 years (Opelz, et al., Lancet, 1:1223 (1981); Gjertson, UCLA Tissue Typing Laboratory, p. 225 (1992); Powles, Lancet, p. 327 (1980); and Ramsay, New Engl. J. Med., p. 392 (1982)). It would therefore be a major advance if tolerance to the donor cells can be induced in the recipient.

The only known clinical condition in which complete systemic donor-specific transplantation tolerance occurs is when chimerism is created through bone marrow transplantation (Qin, et al., J. Exp. Med., 169:493-502 (1989); Sykes, et al., Immunol. Today, 9:23-27 (1988) and Sharabi, et al., J. Exp. Med., 169:779 (1989)). This has been achieved in neonatal and adult animal models as well as in humans by total lymphoid or body irradiation of a recipient followed by bone marrow transplantation with donor cells. The success rate of allogeneic bone marrow transplantation is, in large part, dependent on the ability to closely match the major histocompatibility complex (MHC) of the donor cells with that of the recipient cells to minimize the antigenic differences between the donor and the recipient, thereby reducing the frequency of host-versus-graft responses and graft-versus-host disease (GVHD). In fact, MHC matching is essential; only one or two antigen mismatch is acceptable because GVHD is very severe in cases of greater disparities.

The field of bone marrow transplantation was developed originally to treat bone marrow-derived cancers. It is believed by those skilled in the art even today that lethal conditioning of a human recipient is required to achieve successful engraftment of donor bone marrow cells in the recipient. In fact, prior to the present invention, current conventional bone marrow transplantation has exclusively relied upon lethal conditioning approaches to achieve donor bone marrow engraftment. The requirement for lethal irradiation of the host, which renders it totally immunocompetent, poses a significant limitation to the potential clinical application of bone marrow transplantation to a variety of disease conditions, including solid organ or cellular transplantation, sickle cell anemia, thalassemia and aplastic anemia.

The risk inherent in tolerance-inducing conditioning approaches must be low when less toxic means of treating rejection are available or in cases of morbid, but relatively benign conditions. In addition to solid organ transplantation, hematologic disorders, including aplastic anemia, severe combined immunodeficiency (SCID) states, thalassemia, diabetes and other autoimmune disease states, sickle cell anemia, and some enzyme deficiency states, may all significantly benefit from a nonlethal preparative regimen which would allow partial engraftment of allogeneic or even xenogeneic bone marrow to create a mixed host/donor chimeric state with preservation of immunocompetence and resistance to GVHD. For example, it is known that only approximately 40% of normal erythrocytes are required to prevent an acute sickle cell crisis (Jandle, et al., Blood, 18(2) (1961); Cohen, et al., Blood, 18(2):133 (1961) and Cohen, et al., Blood, 76(7) (1984)), making sickle cell disease a prime candidate for an approach to achieve mixed multilineage chimerism. Although the morbidity and mortality associated with the conventional full cytoreduction currently utilized for allogeneic bone marrow transplant cannot be justified for relatively benign disorders, the induction of multilineage chimerism by a less aggressive regimen certainly remains a viable option. Moreover, the use of bone marrow from an HIV-resistant species offers a potential therapeutic strategy for the treatment of acquired immunodeficiency syndrome (AIDS) if bone marrow from a closely related species will also engraft under similar non-lethal conditions, thereby producing new hematopoietic cells such as T cells which are resistant to infection by the AIDS virus.

A number of sublethal conditioning approaches in an attempt to achieve engraftment of allogeneic bone marrow stem cells with less aggressive cytoreduction have been reported in rodent models (Mayumi and Good, J. Exp. Med., 169:213 (1989); Slavin, et al., J. Exp. Med., 147(3):700 (1978); McCarthy, et al., Transplantation, 40(1):12 (1985); Sharabi, et al., J. Exp. Med., 172(1):195 (1990) and Monaco, et al., Ann. NY Acad. Sci., 129:190 (1966)). However, reliable and stable donor cell engraftment as evidence of multilineage chimerism was not demonstrated, and long-term tolerance has remained a question in many of these models (Sharabi and Sachs, J. Exp. Med., 169:493 (1989); Cobbold, et al., Immunol. Rev., 129:165 (1992); and Qin, et al., Eur. J. Immunol., 20:2737 (1990)). Moreover, reproducible engraftment has not been achieved, especially when multimajor and multiminor antigenic disparities existed.

Permanent tolerance to donor antigens has been documented in H-2 (MHC) identical or congenic strains with minimal therapy and/or transplantation of donor skin drafts or splenocytes alone (Qin, et al., Eur. J Immunol., 20:2737 (1990)). However, similar attempts to achieve engraftment and tolerance in MHC-mismatched combinations have not enjoyed the same success. In most models, only transient donor-specific tolerance has been achieved (Mayumi, et al., Transplantation, 44(2):286 (1987); Mayumi, et al., Transplantation, 42(4):286 (1986); Cobbold, et al., Eur. J Immunol., 20:2747 (1990); and Cobbold, et al., Seminars in Immunology, 2:377 (1990)).

Early work by Wood and Monaco attempted to induce tolerance using bone marrow plus anti-lymphocyte serum (ALS) in partial MHC-matched donor-recipient combinations (Wood, et al., Trans. Proc., 3(1):676 (1971); and Wood and Monaco, Transplantation, 23:78 (1977)). Even in this semi-allogeneic system, F1 splenocytes were required to facilitate the induction of tolerance, and thymectomy was required for stable long-term tolerance. The additional requirement for splenocytes and thymectomy made potential clinical applicability of such an approach unlikely. However, these studies identified two key factors required for induction of tolerance: an antigenic source of tolerogen, which is not only involved in tolerance induction, but must also be present at least periodically for permanent antigen-specific tolerance, and a method to tolerize or prevent activation of new T cells from the thymus, i.e. thymectomy, or intrathymic clonal deletion.

Attempts to induce tolerance to allogeneic bone marrow donor cells using combinations of depleting and non-depleting anti-CD4 and CD8 monoclonal antibodies (mAb) resulted in only transient tolerance to MHC-compatible combinations (Cobbold, et al, Immunol. Rev., 129:165 (1992); and Qin, et al., Eur. J. Immunol, 20:2737 (1990)). 6 Gy of TBI was required to obtain stable engraftment and tolerance when MHC-disparate bone marrow was utilized (Cobbold, et al., Transplantation, 42:239 (1986)). Sharabi and Sachs attributed the failure of anti-CD4/CD8 mAb therapy alone to the inability of mAb to deplete T cells from the thymus, since persistent cells coated with mAb could be identified in this location (Sharabi and Sachs, J. Exp. Med., 169:493 (1989)). However, subsequent attempts to induce tolerance by the addition of 7 Gy of selective thymic irradiation prior to donor bone marrow transplantation also failed. Engraftment was only achieved with the addition of 3 Gy of recipient TBI.

The cells of all hematopoietic lineages are produced by hematopoietic stem cells (HSC). During this procedure, some HSC retain a long-term multilineage repopulating potential (self-renewal); and some HSC may only retain a short-term multilineage repopulating potential and differentiate to produce progeny (Allcock, R. J., et al, Immunol. Today, 21:328-332 (2000)). The major purified HSC transplantation-related complications include graft rejection and graft failure. The outcome for engraftment of highly purified HSC in the major histocompatibility complex (MHC)-matched recipients is different from that for MHC-disparate allogeneic recipients (Bix, M., et al., Nature, 349:329-331 (1991); and Burt, R. K, et al., Stem Cells, 17:366-372 (1999)).

The major histocompatibility complex is a cluster of closely linked genetic loci encoding three different classes (class I, class II, and class III) of glycoproteins expressed on the surface of both donor and host cells that are the major targets of transplantation rejection immune responses. The MHC is divided into a series of regions or subregions and each region contains multiple loci. An MHC is present in all vertebrates, and the mouse MHC (commonly referred to as H-2 complex) and human MHC (commonly referred to as the Human Leukocyte Antigen or HLA) are the best characterized.

The role of MHC was first identified for its effects on tumor or skin transplantation and immune responsiveness. MHC molecules are cell surface receptors that bind antigen fragments and display them to various cells of the immune system, most importantly T cells that bear αβ receptors Natural Killer (NK) cells λδ-T cells. Different loci of the MHC encode two general types of antigens which are class I and class II antigens. In the mouse, the MHC consists of 8 genetic loci: class I is comprised of K and D, class II is comprised of I-A and/or I-E. The class II molecules are each heterodimers, comprised of I-Aα and I-Aβ and/or I-Eα and I-Eβ. One major function of the MHC molecule in immune recognition is to provide restriction by binding of peptides and the interaction with T cells, usually via the T-cell receptor for antigen processing and presentation. For example CD8 positive T cells that develop in a recipient recognize antigen-presenting cells (APC) expressing class I host-type MHC, a process termed “restriction.” More recently, a role for class I MHC functions in CNS development by engaging CD3I-containing receptors to signal activity dependent changes in synaptic strength that ultimately lead to the establishment of appropriate synapses has been demonstrated.

Transplantation of purified HSC across allogeneic barriers encounters greater host resistance, resulting in higher incidences of graft failure (Bix, M., et al., Nature, 349:329-331 (1991); Hayashi, H., et al., Bone Marrow Transplant, 18:285-292 (1996); and Ildstad, S.T., et al., Nature, 307:168-170 (1984)). The mechanism underlying this observation has remained undefined, if the HSC donor and recipient are MHC-congeneic, irrespective of the minor antigen matching, long-term engraftment of HSC occurs reliably. In striking contrast, if donor and recipient are MHC-disparate, readily and only short-term radioprotection is observed, even when syngeneic marrow is co-administered concomitantly. This graft failure has been attributed to NK-mediated rejection. However, the kinetics for graft failure differ significantly from the rapid NK-mediated rejection observed in bone marrow transplant from class I deficient donors.

When small numbers of unmodified bone marrow cells are administered, allogeneic HSC engraft in relatively small numbers. Similarly, if CD8⁺/TCR⁻ facilitating cells (FC) are co-administered with similar numbers of purified HSC, engraftment is restored in MHC-disparate allogenic recipients. The biologic effect of graft facilitation occurs only if the FC is MHC-congenic to the HSC. There remains a continuing need to determine which molecules will facilitate engraftment and self-renewal of HSC. There is also a further there remains a need for non-lethal methods of conditioning a recipient for allogeneic bone marrow transplantation that would result in stable mixed multilineage allogeneic chimerism and long-term donor-specific tolerance. And ultimately to define the specific cells that are needed without conditioning.

### SUMMARY OF THE INVENTION

Accordingly, one aspect of the present invention is to evaluate the role of MHC class I and class II molecules in engraftment of purified HSC in allogenic recipients disparate at specific loci.

Another aspect of the present invention is to determine whether or not facilitating cells and HSC must be genetically matched at specific MHC loci for facilitation to occur in MHC disparate recipients.

The present invention further provides a method for significantly decreasing the rate of host resistance to the transplantation of purified hematopoietic stem cells across allogeneic barriers thereby resulting in lower incidences of graft failure.

The present invention further provides a method for producing a chimeric cell population wherein the major histocompatibility complex is specifically matched at a loci.

More specifically, one method of this invention comprises achieving a higher rate of allogeneic hematopoietic stem cell engraftment by either (i) matching the major histocompatibility complex class I K locus between donors and recipients or (ii) identifying how class I K on HSC interact with FC (CD8/33Kd receptor complex) works thus allowing one to bypass the need for FC.

Additional advantages, and novel features of this invention shall be set forth in part in the description and examples that follow, and in part will become apparent to those skilled in the art upon examination of the following or may be learned by the practice of the invention. The advantages of the invention may be realized and attained by means of the instrumentalities and in combinations particularly pointed out in the appended claims.

### BRIEF DESCRIPTION OF THE DRAWINGS

The accompanying drawings, which are incorporated in and form a part of the specifications, illustrate the preferred embodiments of the present invention, and together with the description serve to explain the principles of the invention.

In the Drawings:

FIG. 1 demonstrates, through shading, the MHC-disparity relative to B10.BR.

FIG. 2 is a Kaplan-Meier survival curve of recipients of 5000 syngeneic (B10.BR→B10.BR), MHC congenic minor plus antigen disparate (B10.BR→AKR), and MHC-disparate minor antigen congenic (B10.BR→C57BL/10) HSC following conditioning with 950 cGy TBI.

FIG. 3 demonstrates, through shading, the MHC-disparity relative to B10.BR.

FIG. 4 is a Kaplan-Meier curve for mice conditioned with 950 cGy TBI and transplanted with 5000 B10.BR HSC.

FIG. 5 is a Kaplan-Meier curve that compares survival of recipients of HSC disparate at class I K plus class I D (B10.BR→B10.MBR) versus class I K only.

FIG. 6 is an analysis of mixed chimeras by flow cytometry.

FIG. 7 in an analysis of mixed chimeras by flow cytometry, illustrating that donor class II I-E is represented in these chimeras.

FIG. 8 illustrates the reactivity of mixed allogeneic chimeras (B 10.A→B10 A 4R) in MLR assay.

FIG. 9 shows 5000 MSC and 30,000 FC sorted from donors disparate at selected MHC loci, mixed, and transplanted into BIO recipients. The shading in FIG. 9 shows the disparity between FC donor and B 110.BR HSC donor.

FIG. 10 is a Kaplan-Meier Curve the figure legend represents the strain of HSC donor, FC donor, and disparity between the HSC and FC donor.

FIG. 11 shows the percent donor chimerism versus time and absolute WBC at 180 days for 5000 MSC and 30,000 FC sorted from donors disparate at selected MHC loci, mixed, and transplanted into B 10 recipients.

FIG. 12 represents graphically an assessment of mixed chimerism by flow cytometry. PBL from HSC and FC recipients were stained with specific MHC class I antigen of donor and recipients and the percentage donor chimerism enumerated monthly.

### DETAILED DESCRIPTION OF THE PREFERRED EMBODIMENT

It has been discovered that class I K are essential molecules for engraftment of allogeneic hematopoietic stem cells (HSC), since disparate at major histocompatibility complex (MHC) class I K locus between donor and recipient, impaired engraftment results. Conversely, with matching at class IK, successful engraftment was achieved. It was further discovered that facilitating cells (FC) are critical for engraftment of purified HSC in allogeneic recipients, since 100% animals of FC plus HSC exhibited durable mixed chimerism and long-term survival. When FC and HSC are matched at the class I K locus, FC exhibit a greater ability to facilitate engraftment of allogeneic HSC, suggesting that MHC class I K is an important molecule involved in the direct interaction between FC and HSC. The data discussed below in detail provide the first evidence that MHC class I K is an important molecule to influence engraftment of allogeneic HSC.

The present invention is discussed in more detail below, solely for the purpose of description and not by way of limitation. For clarity of discussion, the specific procedures and methods described herein are exemplified using a murine model; they are merely illustrative for the practice of the invention. Analogous procedures and techniques are equally applicable to all mammalian species, including human subjects.

To evaluate which MHC loci were important to HSC engraftment, mice congenic at various loci were utilized as recipients. Mice of different strains provide a reasonable model to study the role of MHC loci on engraftment or graft failure due to different MHC loci and genetic backgrounds (Kaufman, C. L., et al., Blood, 84:2436-2446 (1994); Lechler, R., et al., Curr. Opin. Immunol., 3:715-721 (1991); Lowin-Kropf, B., et al., J. Immunol., 165:91-95 (2000); and Meyer, D., et al., Immunobiology, 197:494-504 (1997)).

The mouse strain combinations tested included MHC-match, minor histocompatibility, major plus minor histocompatibility mismatches, MHC-class I or class II disparate and MHC class I or class II deficient. The strain combinations were chosen so that donor and recipient hematopoietic cell contribution could be distinguished at the MHC locus. HSC are defined by the following combination of cell surface markers: Sca-1⁺/C-kit⁺/Lin⁻. Cells with this phenotype have been found to contain a population of cells with long-term multilineage reconstitution potential. (Allcock, R. J., et al., Immunol. Today, 21:328-332 (2000); Bix, M., et al., Nature, 349:329-331 (1991); Ohlen, C., et al., Eur. J Immunol., 25:1286-1291 (1995); Schuchert, M. J., et al., Nat. Med., 6:904-909 (2000); Shenoy, S., et al., Clin. Exp. Immunol., 112:188-195 (1998); and Shizuru, J. A., et al., Biol. Blood Marrow Transplant., 2:3-14 (1996)). The data discussed in detail below, demonstrate that the purified HSC engraft readily in MHC-match (BR→BR) or minor antigen disparate recipients (BR→AKR), but not in fully MHC-disparate recipients (BR→B10). Highly purified HSC in MHC-disparate recipients allow prolonged survival. However, all animals expire within 180 days due to marrow aplasia and late graft failure. These results suggest that committed progenitor cells (that are no longer self-renewing HSC) survival and function for up to 180 days (Ildstad S.T., Transplantation Science, 3:123 (1993)).

Previous studies have indicated that B6 β2m−/− (class I deficient) mice marrow did not engraft in MHC-matched (C57BL/6x129) F₂ normal mice after lethal radiation of recipients, suggesting that rejection of class I-deficient cells is mediated by normal NK cells (Domen, J., et al., J. Exp. Med., 191:253-264 (2000); Grigoriadou, K., et al., Eur. J. Immunol., 29:3683-3690 (1999); Lowin-Kropf, B., et al., J. Immunol., 165:91-95 (2000); Spangrude, G. J., et al., Science, 241:58-62 (1998); Spangrude, G. J, et al., Blood, 78:1395-1402, (1991); Stoltze, L., et al., Today, 21:317-319 (2000); Uchida, N., et al., J. Clin. Invest., 101:961-966 (1998); Ugolini, S., et al., Curr. Opin. Immunol., 12:295-300 (2000); and Vallera, D. A., et al., Transplantation, 57:249-256 (1994)). The data in this application shows that all donor B6 β2m (class I deficient) HSC failed to engraft in B6 mice, while all Abb (class II deficient) HSC engrafted in B6 mice, strongly suggesting that the molecules of MHC class I contribute to engraftment.

To determine which MHC molecule is required for HSC engraftment, mice matching at certain MHC loci but disparate at other loci were tested. Inbred mouse strain combinations congenic for all except specific MHC class I and class II loci were utilized as recipients. Again, the data discussed in detail below demonstrate that MHC class I D is not essential for HSC engraftment since 100% animals engrafted in B10.BR→B10.A (2R) combinations and survival over 180 days. However, if the MHC-disparate at class I K locus in B10.BR→B10.MBR combinations, 17% animals engrafted of HSC and survival over 180 days. Therefore, class I K is important to HSC engraftment. Furthermore, in mice transplanted across the MHC-disparate class I K and class II I-A loci (B10.BR→B10.A (5R)), animals show poor engraftment of HSC, about 25% animal survival over 180 days. Further, indicating importance of class I K and possibly class II IA in HSC engraftment. In striking contrast, if the donor and recipient are matched at class I K and class II IA in B10.BR→B10.A (4R), 83% animals show long-term survival over 180 days and exhibited durable mixed chimerism of all the lymphoid (T and B lymphocytes), NK, and myeloid (macrophages, granulocytes) cell populations. Moreover, chimeras exhibited donor-specific tolerance in vitro.

In a previous study, it was shown that FC (CD8⁺/TCR⁻) promotes allogeneic HSC engraftment across major and minor histocompatibility complex barriers without causing GVHD. When the addition of FC plus HSC was administered to allogeneic recipients, successful engraftment resulted, and animals exhibited stable multilineage chimerism and donor-specific transplantation tolerance (Bix, M., et al., Nature, 349:329-331 (1991)). The data presented herein shows that by transplanting 5000 purified HSC plus 30,000 FC from donor B10.BR mice into lethally irradiated, MHC-disparate allogeneic B10.A (5R), B10.MBR, C57BL/10 and B10.A (4R) recipients, 100% of the animals engrafted and exhibited long-term survival with durable mixed chimerism. These results strongly demonstrated that the FC is critical for engraftment of HSC in MHC-disparate recipients.

The mechanism of FC (CD8⁺/TCR⁻) population enhances engraftment of allogeneic HSC may be related to that of HSC expression at the MHC loci. It is hypothesized that the FC influences survival of HSC by direct interaction. Consequently, it was further determined which MHC locus requires recognition of FC. The data presented herein shows that 100% of the animals engrafted if there were HSC and FC matching at MHC class I K locus. In contrast, 50% to 62% of the animals engrafted if there was HSC and FC mismatching at H-2 or MHC I K. These data suggest that receptor-MHC Ligand interaction plays a dominant effect.

The data indicate that recipient and donor matching at the class I D is not essential for HSC engraftment. Moreover, matching at MHC class II I-E is not essential for HSC engraftment when I-E is not expressed. In striking contrast, MHC disparate at the class I K locus results in significantly impaired engraftment of HSC. The addition of as few as 30,000 facilitating cells (CD8⁺/TCR⁻) can restore engraftment of HSC in allogeneic recipients without causing GVHD. Further, if facilitating cells and HSC match at the MHC class I K, facilitating cells have a strong biologic effect on engraftment in allogeneic recipients. These results demonstrate that MHC class I K is an essential molecule for engraftment of allogeneic HSC. The method of this invention achieves a higher rate of allogeneic hematopoietic stem cell engraftment by either (i) matching the major histocompatibility complex class I K locus between donors and recipients or (ii) identifying how class I K on HSC interact with FC (CD8/33Kd receptor complex) works thus allowing one to bypass the need for FC.

The following non-limiting examples provide methods for enhancing durable engraftment of purified HSC in allogeneic recipients by matching the MHC class I K. All scientific and technical terms have the meanings as understood by one with ordinary skill in the art. The methods may be adapted to variation in order to produce compositions or devices embraced by this invention but not specifically disclosed. Further variations of the methods to produce the same compositions in somewhat different fashion will be evident to one skilled in the art.

The examples that follow demonstrate the utility of the present invention by clearly exemplifying the underlying discovery that the MHC class I K molecule is essential for maintaining the self-renewal capability of purified HSC. Moreover, interaction between the HSC and FC via the MHC class I K molecule provides a regulatory function to promote engraftment and survival of allogeneic HSC.

### EXAMPLES

### Materials and Methods

Mouse strains

Four to 5-week-old male B10.BR, AKR, C57BL/10, B10.MBR, B10.A (2R), B10.A (4R), B10.A (5R), and BALB/c mice were purchased from the Jackson Laboratory (Bar Harbor, Me.). C57BL/6, C57BL/6-β2m (MHC class I deficient) and C57BL/6Abb (MHC class II deficient) mice were purchased from the Taconic (Germantown, N.Y.). Animals were housed in a barrier animal facility at the Institute for Cellular Therapeutics, University of Louisville, Louisville, Ky., and cared for according to specific University of Louisville and National Institutes of Health animal care guidelines.

Antibodies

All of the monoclonal antibodies (mAbs) used in this study were purchased from Pharmingen. Stem cell sorting experiments used directly conjugated mAbs and include stem cell antigen-1 PE (E13-161.7; rat IgG₂ₐ), c-kit APC (2B8; rat IgG_{2b}), CD8α FITC (53-6.7; rat IgG₂ₐ), Mac-1 FITC (M1/70; rat IgG_{2b}), B220 FITC (RA3-6B2; rat IgG₂ₐ), Gr-1 FITC (11-26c.2a; rat IgG₂ₐ), β-TCR FITC (H57-597; armenian hamster IgG). Facilitating cell sorting experiments used β-TCR FITC (H57-597; armenian hamster IgG); γδ-TCR FITC (GL3; armenian hamster IgG); and CD8α PE (53-6.7; rat IgG₂ₐ). H-2K^{k} FITC (AF3-12.1; mouse IgG¹), H-2K^{b} PE (AF6-88.5; mouse IgG₂ₐ); H-2D^{d} PE (34-2-12; mouse IgG₂ₐ), and H-2D^{b} PE (KH95; mouse IgG_{2b}) mAbs were used for assessment of chimerism.

Purification of Hematopoietic Stem Cell (Sca⁺/C-kit⁺/Lin⁻) and Facilitating Cells (CD8⁺/TCR⁻)

Populations were positively selected from bone marrow using a multiparameter, live sterile cell sorter (FACS Vantage SE; Becton Dickinson). Hematopoietic stem cells or facilitating cells were prepared as previously described (Re. Blood). Briefly, bone marrow was isolated and resuspended in a single cell suspension at a concentration of 100×10⁶ cells/ml in 1 mL of sterile cell sort media (CSM), which contains sterile 1× Hank's Balanced Salt Solution without phenol (GIBCO), 2% heat-inactivated fetal calf serum (FCS; GIBCO), 10 mM/mL 1× HEPES buffer (GIBCO), and 30 μL/mL of Gentamicin (GIBCO). Directly labeled mAbs were added at saturating concentrations and the cells were incubated for 30 minutes and washed twice. Cells were resuspended in CSM at 2.5×10⁶ cells/mL. All cells and collecting tubes were maintained on ice during the sorting process.

Hematopoietic Stem Cells Transplantation

Donors and recipients were chosen based on MHC-matching, minor antigens-disparities and MHC-disparities at different loci. Recipient AKR, B10.A (2R) B10.A (4R), B10.A (5R), B10.MBR, C57BL/10, C57BL/6-β2m (class I deficient), C57BL/6-Abb (class II deficient) and BlO.BR mice were conditioned with 950 cGy total body irradiation (TBI) and reconstituted with 5000 purified HSC of donor BIO.BR mice by tail vein injection. The following allogeneic strain combinations were tested including: B10.BR→AKR (MHC minor antigens-disparate); B10.BR→C57BL/10 (disparate at H-2); B10.BR→C57BL/6-β2m (MHC class II disparate with class I deficient); B10.BR→C57BL/6-Abb (MHC class I disparate with class II deficient); B-10.BR→B10.A (2R) (MHC class I D disparate); B10.BR→B10.MBR (MHC class I K and D disparate); B10.BR→10.A (4R) (MHC class I D and no class II I-E expression); B10.BR→B10.A (5R) MHC class I K, D and class II I-A disparate). The syngeneic strain combination B10.BR→B10.BR serves as the control.

Assessment of Chimerism

Thirty days post HSC transplantation, recipients were characterized for allogeneic engraftment using two-color-flow cytometry. Chimerism was determined measuring the percentage of peripheral blood lymphocyte (PBL) of donor (B10.BR) or recipients (B10.A [2R], B10.A [4R], B10.A [5R], B10.MBR and C57BL/10) MHC class I antigen. Briefly, whole blood from recipients was collected in heparinized tubes, and aliquots of 100 μL were stained with anti-H-2K^{k}-FITC and/or anti-H-2K^{b}-PE, anti-H-2D^{d}-PE, anti-H-2D^{b}-PE for 30 minutes. Red blood cells were lysed with ammonium chloride lysing buffer for 5 minutes at room temperature, then washed twice in FACS medium and fixed in 1% paraformaldehyde.

Spleens from mixed allogeneic chimeras were analyzed 6 months following reconstitution for donor and host lymphoid (T and B cell), NK, and myeloid (macrophage and granulocyte) lineages. Briefly, spleens were individually crushed using a sterile glass stopper and washed before staining with mAbs for 30 minutes at 4° C. Lineage typing was performed by two-color flow cytometry using anti-B cell (B220), T-cell (αβ-TCR, CD4, and CD8), granulocyte (Gr-1), monocyte/macrophage (Mac-1) and NK cell (NK1.1) FITC mAbs. Lineage-specific mAbs conjugated to PE was used to anti-donor (H-2K^{k}) and anti-host (H-2D^{b}). Analyses were performed using forward and side scatter characteristic for the lymphoid and myeloid gates. An isotype control as used as background staining.

Proliferation Assay

Splenocytes of naive or chimeric mice were used as responders in a standard mixed lymphocyte reactions (MLR) assay (Ohlen, C., et al., Science, 246:666-668 (1989)). Briefly, a single cell suspension was prepared from spleens in complete MLR medium consisting of DMEM (Life Technologies), supplemented with 1 mM sodium pyruvate, 10 mM HEPES, 100 μL/mL penicillin, 100 μg/mL streptomycin, 0.137 M L-arginine, 1.36 mM folic acid, 50 μM 2-β mercaptoethanol, 12 mM L-gutamine, 5% fetal bovine serum, and 1% normal mouse serum. Splenocytes were used as stimulators after irradiation at 2000 cGy in the Gammacell irradiator (Gammacell® 1000 Elite, Nordion International Inc., Ontario, Canada). Responder and stimulator cells were co-cultured in triplicates at a cell concentration of 5×10⁵ cells/well in 200 μL of complete MLR medium in a 96-well U-bottom microtiter plate (Corning Glass Works, Corning, N.Y.). Cultures were incubated at 37° C. in a 5% CO₂ incubator for 4 days. Responses to irradiated B10.BR and BALB/c splenocytes served as autologous and allogeneic controls. Cells were pulsed with 1 μCi of ³H-Thymidine (NEN Life Sciences Products, Boston, Mass.) for the last 18 hours of the culture period. Cultures were then harvested using the β-plate harvester (TOMTEC Harvester 96, Gaithersburg, Md.) and ³H-Thymidine indine incorporation was determined using a scintillation counter (1205 Betaplate, Wallac Inc.). All MLR assays were performed in 3 replicate wells per data point, and results are presented as mean±SD of triplicate wells of representative experiments. Hematopoietic stem cells plus facilitating cells (CD8+/TCR-) transplantation.

HSC and FC were sorted from mice of the same strain. Recipient B10.A (4R), B10.A (5R), B10.MBR, and C57BL/10 mice were conditioned with 950 cGy of TBI and reconstituted with 5000 HSC and 30,000 FC from donor B10.BR mice by tail vein injection. Recipient C57BL/10 were transplanted with 30,000 FC alone as a control.

Statistical Analysis

Experimental data were evaluated for significant differences using the Independent-Samples t test; p<0.05 was considered a significant difference. Graft survival was calculated according to the Kaplan-Meier method.

Class I K is Essential Molecule for Engraftment of Purified Allogeneic HSC

Matching between recipient and donor HSC at class I K is critical to durable HSC engraftment and self-renewal, while matching at class II and/or class I D is not. Moreover, the co-administration of as few as 30,000 FC congeneic at class I K to the HSC restores engraftment of purified HSC in completely MHC-disparate allogeneic recipients. In the absence of class I K matching between HSC and recipient or HSC and FC, recipients of purified HSC expire from late graft failure with 6 months. Taken together, these data demonstrate that class I K is an essential molecule for engraftment and self-renewal of allogeneic HSC and contributes to regulation of HSC self-renewal.

### Example 1

### Class I Matching is Critical to Engraftment of Purified HSC in Allogeneic Recipients

To determine which genetic loci are important to engraftment of HSC, recipient B10.BR, AKR, C57BL/10, B10.A (2R), B10.A (4R), B10.A (5R) and B10, MBR mice were conditioned with 950 cGy and transplanted with 5000 Sca-1⁺/c-kit⁺/lineage⁻ HSC from B10.BR donors (Table 1).

TABLE I
MHC class I and class II loci between donor and recipient mice
H-2 complex

| Mouse strain | K | Aβ | Aα | Eβ | Eα | D | Minor Antigen |
|---|---|---|---|---|---|---|---|
| B10.BR | k | k | k | k | k | k | Mls^{b} |
| AKR | k | k | k | k | k | k | Mls^{a} |
| B10.MBR | b | k | k | k | k | q | Mls^{b} |
| B10.A(2R) | k | k | k | k | k | b | Mls^{b} |
| B10.A(4R) | k | k | k/b | k | —* | b | Mls^{b} |
| B10.A(5R) | b | b | b/k | k | k | d | Mls^{b} |
| C57BL/10 | b | b | b | k | —* | b | Mls^{b} |

*Class II I-E is not expressed in this mouse strain

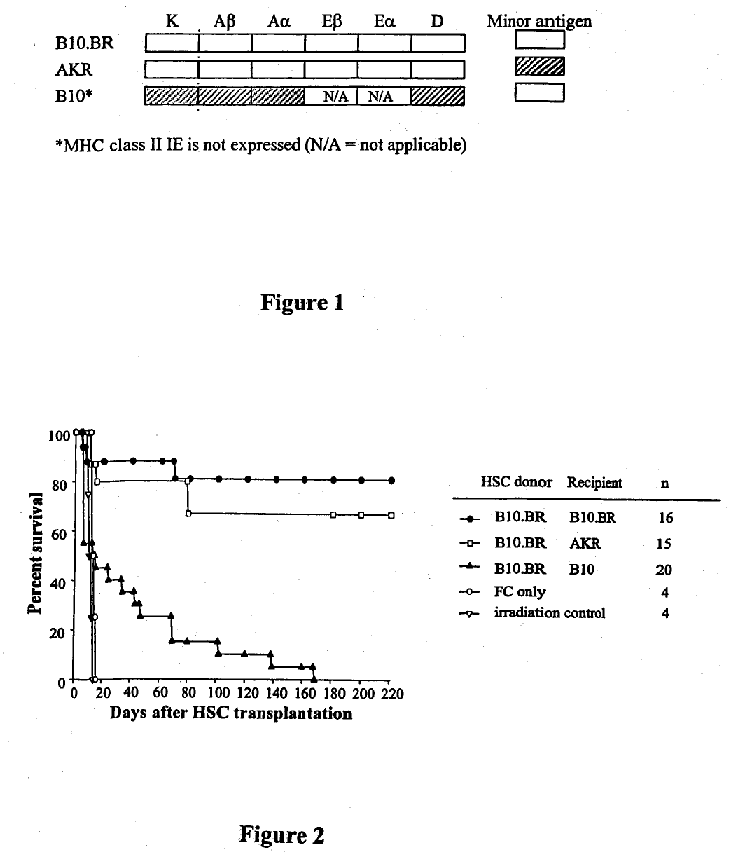

As expected, mice congeneic for MHC (B10.BR→B10.BR) (AKR→B10.BR) exhibited durable engraftment. In striking contrast, as shown in FIGS. 1 and 2 HSC provided short-term radioprotection but did not durably engraft MHC-disparate allogeneic recipients. Survival of recipients of allogeneic HSC alone was significantly prolonged compared with recipients of FC alone, which expired at the time of irradiation controls.

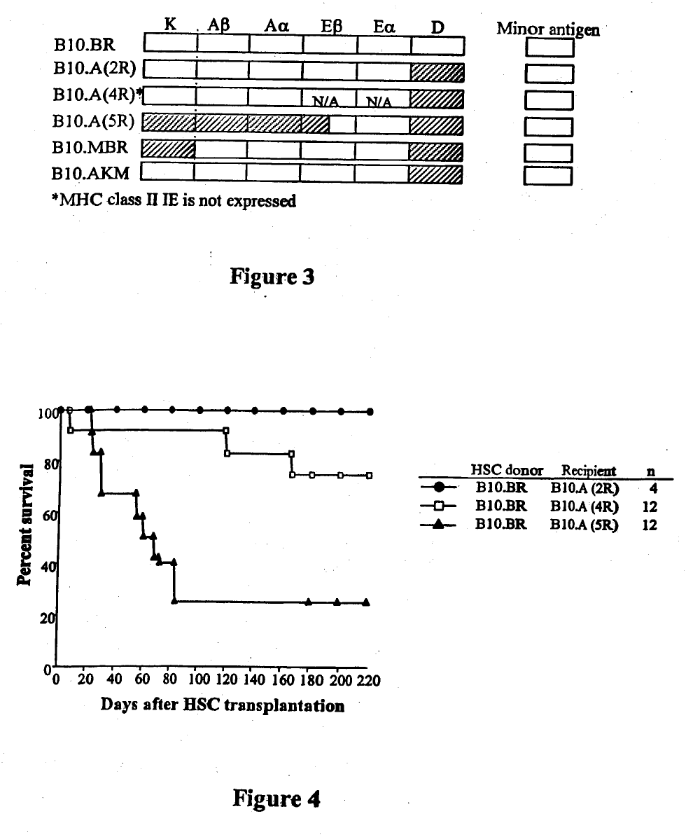

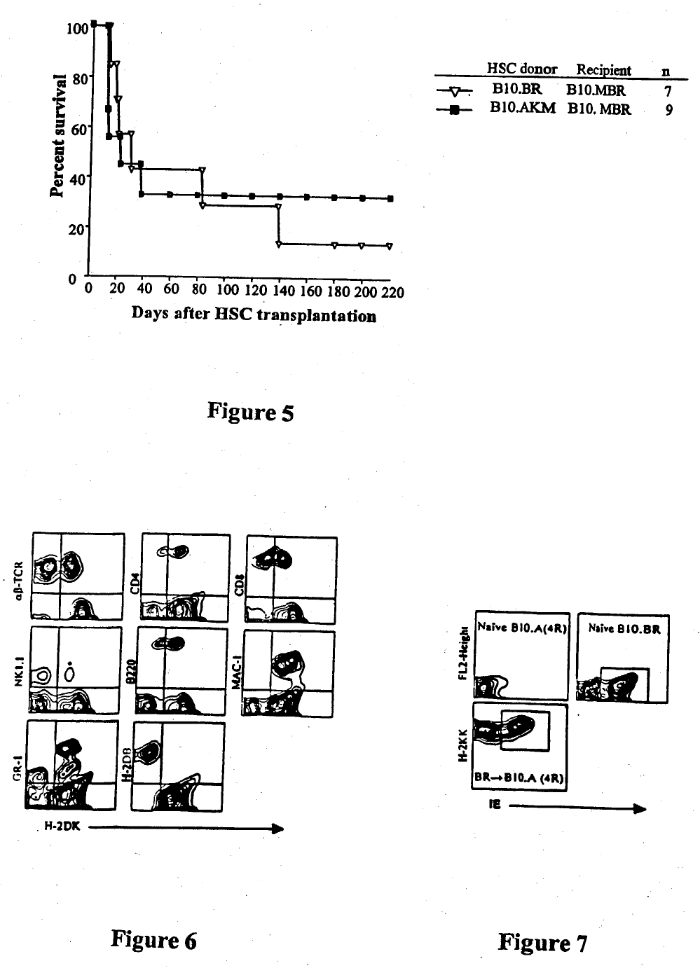

In order to define which MHC loci were important to long-term HSC engraftment and self-renewal, transplants were performed in which specific loci were disparate between HSC donor and recipient. FIG. 3 demonstrates, through shading the MHC-disparity relative to B10.BR. FIG. 4 is a Kaplan-Meier curve for mice conditioned with 950 cGy TBI and transplanted with 5000 B10.BR HSC. Recipients were disparate at class I D (B10.BR→B10.A2R), class I D with no class II I-E expression (B10.BR→B10.A4R), class I K, D and class II I-A (B10.BR→B10.A5R), and class I K plus D (B10.BR→B10.MBR). Sorts of <95% purity were not transplanted. Mice were evaluated monthly for percentage donor and host chimerism and multilineage production. Only three of twelve (25%) of recipients in which the HSC was disparate to the recipient at class I K, D, and class II A survived up to 180 days (B10.BR→B10OA (SR)) and only one of seven (14%) recipients of HSC disparate at class I K and D engrafted (FIG. 4). Class I K disparate HSC offer relative radioprotection compared with radiation controls, but recipients expire from late graft failure up to 180 days following transplantation. In striking contrast, 100% and 83% of recipients of HSC disparate at class I D and class I D in a strain in which there is no class II I-E expression, respectively, engrafted durably. Taken together these data indicate that matching at MHC class I D is not essential for HSC engraftment and self-renewal, nor is matching at class II I-E since I-E is not expressed in B10.A (4R) mice. In striking contrast, if the recipient is disparate at class I K plus class II I-A or class I K plus class I D to the HSC, long-term engraftment of HSC is significantly impaired. To define whether matching at class I K whether matching at class I K was the critical MHC locus, HSC from B10.AKM donors were transplanted into ablated B10.MBR recipients (FIG. 5), a strain combination disparate only at class I K. Although short-term radioprotection was observed, long-term engraftment was significantly impaired. Taken together these data suggest that the MHC class I K molecule is critical for durable engraftment and self-renewal of purified HSC.

Evidence for Multilineage Mixed Chimerism

As discussed previously matching at MHC class I-K is critical for engraftment of allogeneic HSC in B10.BR→B10.A (4R). To determine whether chimeras had evidence of engraftment of the pluripotent stem cell, the proportion of cells within each hematopoietic lineage that were donor B10.BR or host B10.A (4R) derived was enumerated. Animals were tested 6 months following reconstitution. All chimeras analyzed contained cells of donor origin within each of the hematolymphopietic lineages. The presence of donor-derived T lymphocytes, B lymphocytes, NK cells and macrophage/granulocytes was evident as H-2K^{k+}/αβ-TCR⁺, CD4⁺, CD8⁺, B220⁺, Mac-1⁺, Gr-1⁺, and NK1.1⁺ cell populations. A representative example of multilineage chimerism is shown in FIG. 6. FIGS. 6 and 7 are analysis of mixed chimeras by flow cytometry. Splenocytes were stained with the indicated mAbs. FIG. 6 demonstrates that donor B cells, T cells, NK cells, granulocyte and monocytes/macrophage are represented in mixed chimeras (B10.BR→B10A 4R). Expression of the donor B10.BR MHC class II I-E molecule was demonstrated by the presence of an H-2K^{k+}/I-E⁺ cell population in the recipient B10.A (4R), since B10.A (4R) mice do not express this molecule, as shown in FIG. 7.

Donor-Specific Tolerance In Vitro

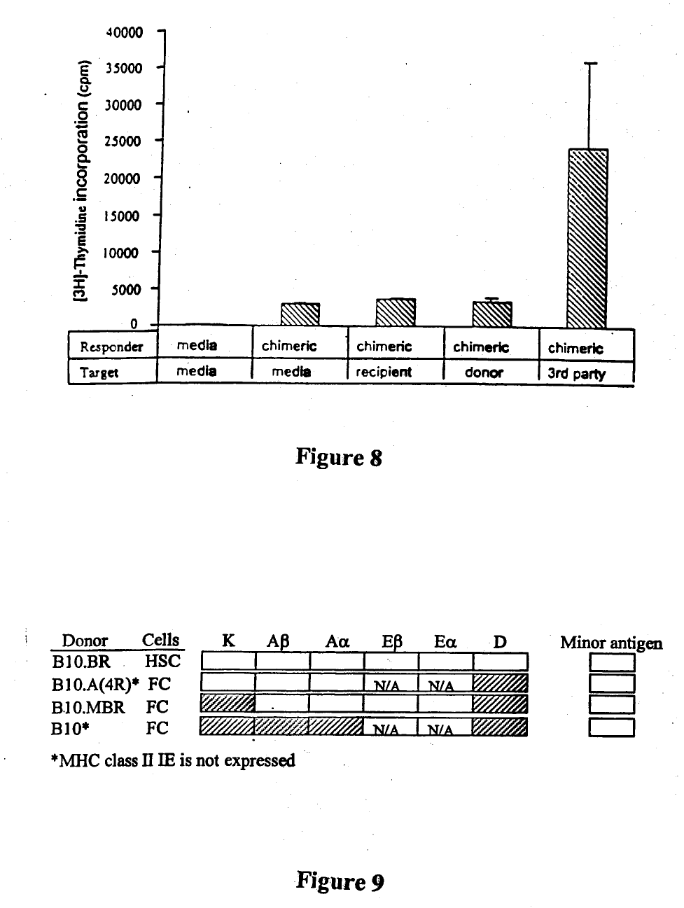

Mixed chimeras B10.BR→B10.A (4R) were tested for evidence of donor-specific tolerance in vitro by using an MLR assay directed against donor and third party antigens. Results are representative of 3 independent experiments are shown in FIG. 8. FIG. 8 represents the reactivity of mixed allogeneic chimeras (B10.A→B10 A 4R) in MLR assay. Stimulator cells of recipient (B10.A 4R), donor (B10.BR), and third party (BALB/c) targets by chimeric splenocytes. This is one of three representative experiments for B10.BR→B10.A 4R chimeras. Splenocytes from chimeras showed a marked reduction in proliferation to donor-specific (B10.BR) stimulator cells compared with naïve responder cells from normal B10.A (4R) mice (p<0.05). These data suggest that chimeras were functionally tolerant to both host and donor alloantigens, but were reactive to MHC-disparate third party alloantigens up to 6 months after reconstitution.

### Example 2

### Facilitating Cells (CD8⁺/TCR⁻) Enhance Engraftment of Allogeneic Hematopoietic Stem Cells: Importance of the MHC class I K Molecule

The facilitating cell is a rare CD8⁺/TCR⁻/CD3ε⁺ cell in bone marrow that enhances engraftment of purified HSC in allogeneic recipients. To determine the role of FC in engraftment and self-renewal of purified HSC in MHC-disparate recipients, HSC and FC obtained from donors and recipients congenic at specific MHC loci were transplanted into MHC-disparate recipients.

As a control, 5000 HSC plus 30,000 FC from BIO.BR donors were transplanted into ablated recipients disparate at class I K and class II (B10.A5R); class I K and D (B10.MBR); and fully MHC-disparate (B10). B10.BR FC alone were transplanted as a control. As expected, recipients of FC alone expired at the time of radiation controls (MST=14 days) (Table 2).

TABLE 2
Result of HSC plus FC Transplantation
% Donor Chimerism (Mean + SD)

| | Engraftment/ | | | | |
|---|---|---|---|---|---|
| Donor → Recipient | n | 30 days | 60 days | 90 days | 120 days |
| B10.BR → B10.A (4R)* | 4/4 | 80.8 ± 6.6 | 79.9 ± 7.2 | 89.6 ± 1.7 | 88.5 ± 3.1 |
| B10.BR → C57BL/10* | 4/4 | 70.6 ± 7.3 | 85.3 ± 3.7 | 94.9 ± 0.6 | 95.2 ± 0.8 |
| B10.BR → B10.A (5R)* | 4/4 | 47.3 ± 39.5 | 82.3 ± 10.2 | 92.9 ± 4.5 | 92.4 ± 4.8 |
| B10.BR → C57BL/10† | 0/4 | | | | |

*Recipients were reconstituted with 5 × 10³ plus 30 × 10³ FC
†Recipients were reconstituted with 30 × 10³ FC; animal dead between 12 and 15 days after transplantation.

All other recipients engrafted and exhibited durable mixed chimerism ≧180. These data further support a mechanism involving direct FC:HSC interaction with additional molecules on the FC cell surface to mediate the biologic effect.

Next, which MHC loci for FC must be matched to HSC for graft facilitation to occur was tested. FC and HSC were sorted from donors disparate at selected MHC loci. Recipient B10 mice were conditioned with 950 cGy TBI and transplanted with 5000 HSC from B10.BR mice and 30,000 FC from B10.A (4R), B10.MBR or C57BL/10 mice.

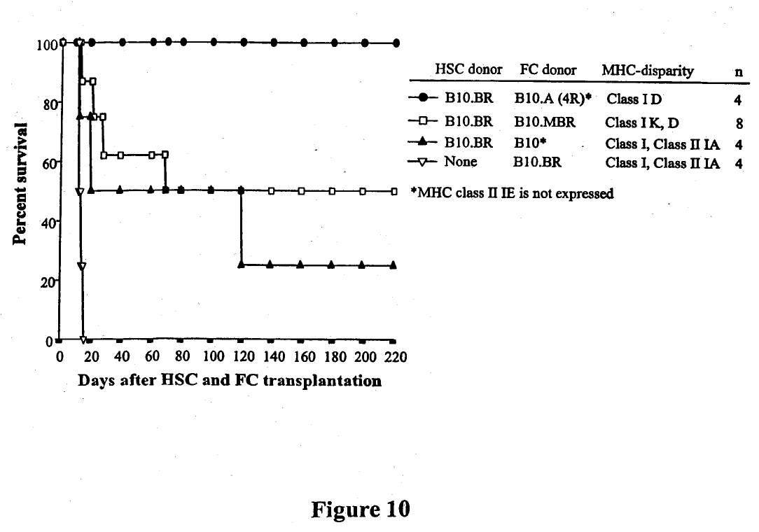

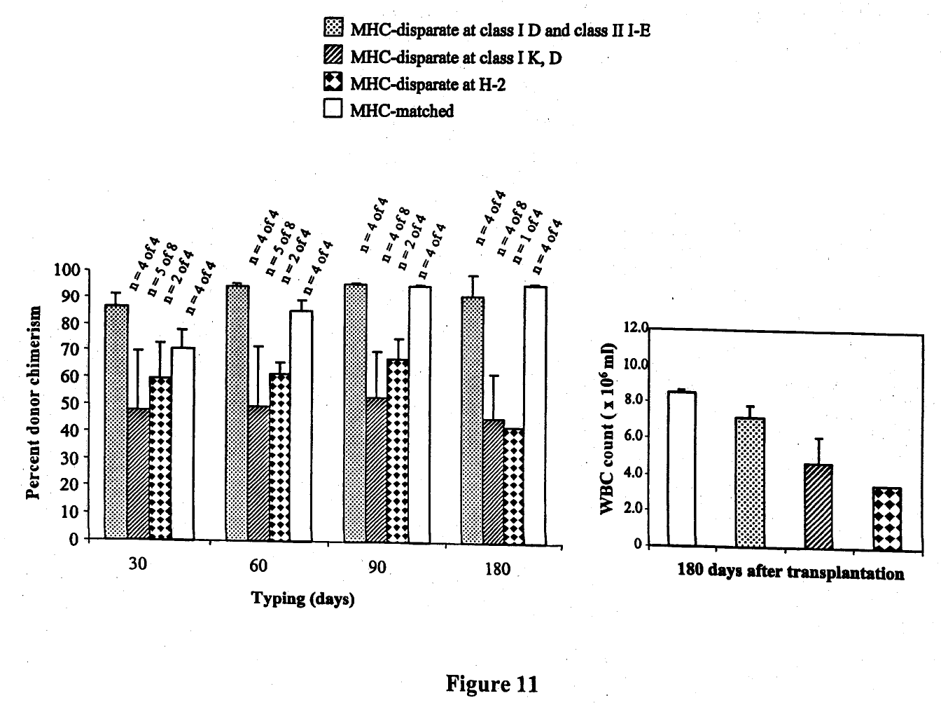

FIGS. 9-11 show 5000 MSC and 30,000 FC sorted from donors disparate at selected MHC loci, mixed, and transplanted into B10 recipients. The shading in FIG. 9 shows the disparity between FC donor and B10.BR HSC donor. FIG. 10 is a Kaplan-Meier Curve the figure legend represents the strain of HSC donor, FC donor, and disparity between the HSC and FC donor. FIG. 11 shows the percent donor chimerism versus time and absolute WBC at 180 days for the four groups. When HSC and FC were MHC-disparate or disparate at the class I K and D locus, 2 of 4 (50%) or 5 of 8 (62%) animals engrafted, respectively. In striking contrast, when the HSC and FC were matched at class I K (B10.BR), 100% of recipients engrafted durably (FIGS. 9 and 10). In serial typing for chimerism, the level of chimerism was higher in proportion in these animals compared with those with an MHC- or class I K locus-disparity between FC and HSC (FIG. 11). B10.BR→B10.A (4R) chimeras exhibit donor-specific tolerance. Also shown in FIG. 11 is the absolute white blood count (WBC) at 180 days for the four groups. This reflects the integrity of the skin graft, that is, as WBC increase in matched FC and HSC without class I matching the group is impaired and HSC self-renewal lost to committed progenitors.

Splenocytes from chimeras were co-cultured in one-way MLR assay with donor or third party alloantigens to evaluate the evidence for donor-specific tolerance. The response to donor alloantigens was markedly reduced compared with MHC-disparate third party (P=0.002), suggesting functional tolerance to donor alloantigens but immunocompetence to respond to third party.

HSC are responsible for steady state continuous production of lineage-committed progenitor cells. HSC are capable of increasing the production of their progeny dramatically in response to various stimuli, including BMT. Despite the dynamic proliferative nature of HSC, the incidence of malignant transformation and bone marrow failure is very low, suggesting that these cells are under very tight regulation. One of the control mechanisms is to prevent HSC from entering the cell cycle. The mechanism by which the hematopoietic microenvironment regulates HSC function and self-renewal has not been defined. There are convincing data to support the fact that all pluripotent HSC undergo intermittent cycling. Moreover, after transplantation, it is hypothesized that HSC must enter into cycle in order to home to the appropriate niche. The hematopoietic microenvironment clearly influences HSC survival and self-renewal. The contribution of MHC molecules to engraftment and self-renewal or lineage commitment has not been evaluated.

The major histocompatibility complex is a genetic region many of whose products are devoted to processing and presentation of antigen to T-lymphocytes, resulting in antigen-specific activation of T cells. Class I is present on most cells of the body and the highest expression is typically on hematopoietic elements. One can consider class I heavy chains to be like deletion mutants that lack a fragment of the wild type sequence required to initiate successful folding and chaperone release intracellularly in the endoplasmic reticulum. It is only after that occurs that peptide is processed and transported to the cell surface to be presented to the T cell for activation of those T cells that recognize that specific peptide as foreign. Interactions between cell surface receptors of APC and T cells are required for T cell activation to result. One could hypothesize that in a system as critical as regulation of HSC survival and function where loss of control could result in malignancy or graft failure, a similar regulatory system may be operational.

Matching between HSC and the hematopoietic microenvironment at class I K plays a critical regulatory role in determining stem cell fate. Murine HSC have been reported to express high levels of MHC class I. The role of this high level expression has not been defined to date. The expression of class I on PHSC remains more controversial. Failure of engraftment of MHC class I-deficient marrow occurs in syngeneic wild type recipients. In striking contrast, bone marrow from class II deficient donors behaves in a fashion similar to that for normal bone marrow donors. These data have been interpreted in the context of bone marrow graft rejection by NK cells. The class I molecule on the target cell is hypothesized to offer partial protection, while certain syngeneic class I molecules provide full protection from NK cell-mediated rejection of bone marrow cells. This data demonstrates that while this mechanism may in part be responsible for the failure of marrow from B2m (−/−) mice to engraft, an alternative hypothesis is that the cascade of events that initiates engraftment and self-renewal of highly purified HSC requires matching or restriction between class I K for the HSC and recipient microenvironment. In the absence of class I K matching between donor and recipient, the HSC is functional to offer relative radioprotection but loses long-term self-renewal capability. The fact that HSC from normal donors lacking class I K matching to the recipient offer short-term radioprotection but also do not durably engraft would support the latter hypothesis, since committed progenitor cells in the mouse can function for up to 6 months.

The facilitating cell CD8⁺/TCR⁻ is a rare event in bone marrow that restores engraftment of highly purified HSC in allogeneic recipients. The FC must be genetically matched to the HSC for the biologic activity to occur. Recently, a unique 33 KD chaperone protein was identified on FC but not control T cells. The addition of FC to purified HSC restores durable engraftment in MHC-disparate allogeneic recipients if the FC and HSC are matched at class I K. Long-term engrafting cells have been demonstrated to undergo cell cycling within 12 hours after transplantation. HSC express some adhesion molecules and primitive markers in a cell-cycle related fashion. It is hypothesized that as HSC exit G₀/G₁ and begin to cycle, that hematopoietic potential may be compromised. It is conceivable that class I K on the HSC contributes to the CD8⁺/TCR-/CD3ε⁺/33kd chaperone protein ligand complex for this receptor in the same way that CD8+T cells are restricted to host MHC class I and that in the absence of FC, purified HSC become committed progenitors.

An alternative explanation for the requirement for class I K matching between HSC and recipient or between FC and HSC would be to prevent NK-mediated lysis. The role of MHC class I and class II in NK cell-mediated rejection of allogeneic, semi-allogeneic, and syngeneic bone marrow grafts has remained controversial. Hematopoietic progenitor cells are sensitive targets for NK cells. The MHC class 1 antigen complex is the critical structure in NK recognition of hematopoietic progenitor cells. This complex mediates resistance of NK-specific lysis of hematopoietic progenitor cells. Molecules encoded by MHC class I are recognized by three distinct groups of cell surface receptors: the TCR, the CD8 dimers, and the NK cell receptors (NKRs). However, NK cells have not been shown to recognize hematopoietic progenitor cells directly. Bone marrow transplanted from B2m−/− donors into ablated allogeneic or semi-allogeneic recipients is rapidly rejected, even when large numbers of cells are administered, with a survival time of 8 to 16 days. The administration of as many as 3×10⁷ (−/−) bone marrow cells fails to radioprotect even short term. Pre-treatment of the recipient with anti-NK mAb enhances short-term engraftment (30 day follow up), implicating NK cells in the rejection process. These data support a mechanism involving direct FC:HSC interaction with additional molecules on the FC cell surface to mediate the biologic effect.

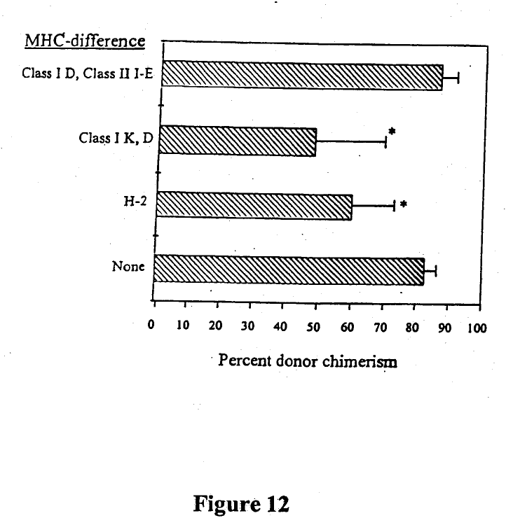

FIG. 12 represents graphically an assessment of mixed chimerism by flow cytometry. PBL from HSC and FC recipients were stained with specific MHC class I antigen of donor and recipients and the percentage donor chimerism enumerated monthly. The percent donor chimerism is expressed as mean±SD. The asterisk indicates P<0.05, which is significantly different from the MHC-matched between HSC and FC mice combinations.

A wide variety of uses are encompassed by the invention described herein, including, but not limited to, the conditioning of recipients by non-lethal methods for bone marrow transplantation in the treatment of diseases such as hematologic malignancies, infectious diseases such as AIDS, autoimmunity, enzyme deficiency states, anemias, thalassemias, sickle cell disease, and solid organ and cellular transplantation.

The foregoing description is considered as illustrative only of the principles of the invention. The words “comprise,” “comprising,” “include,” “including,” and “includes” when used in this specification and in the following claims are intended to specify the presence of one or more stated features, integers, components, or steps, but they do not preclude the presence or addition of one or more other features, integers, components, steps, or groups thereof. Furthermore, since a number of modifications and changes will readily occur to those skilled in the art, it is not desired to limit the invention to the exact construction and process shown described above. Accordingly, all suitable modifications and equivalents may be resorted to falling within the scope of the invention as defined by the claims that follow.

The embodiments of the invention in which an exclusive property or privilege is claimed are defined as follows:

1. A method for conditioning a recipient for bone marrow transplantation comprising subjecting the recipient to treatment with a non-lethal dose of body irradiation, and an alkylating agent followed by transplantation with a donor cell preparation containing hematopoietic stem cells from a donor that are matched at the major histocompatibility complex class I K locus with the recipient hematopoietic microenvironment.

2. The method of claim 1 in which the dose is between 1Gy and 7Gy.

3. The method of claim 1, in which the alkylating agent is cyclophosphamide.

4. A cellular composition comprising mammalian hematopoietic stem cells, which match the recipient hematopoietic microenvironment at the major histocompatibility complex class I K locus.

5. The composition of claim 4, wherein said mammalian hematopoietic stem cells are human.

6. A method of partially or completely reconstituting a mammal's lymphohematopoietic system comprising administering to the mammal the composition of claim 1.

7. The method of claim 6, in which the mammal suffers from autoimmunity.

8. The method of claim 7, in which the autoimmunity is diabetes.

9. The method of claim 7, in which the autoimmunity is multiple sclerosis.

10. The method of claim 7, in which the autoimmunity is sickle cell.

11. The method of claim 7, in which the autoimmunity is anemia.

12. The method of claim 6, in which the mammal suffers from a hematologic malignancy.

13. The method of claim 6, in which the mammal requires a solid organ or cellular transplant.

14. The method of claim 6, in which the mammal suffers from immunodeficiency.

15. A method for decreasing the rate of host resistance to the transplantation of hematopoietic stem cells across allogeneic barriers by matching the major histocompatibility complex class I K locus between the donor and the recipient.

16. A cellular composition comprising mammalian hematopoietic stem cells and facilitating cells that are matched at major histocompatibility complex class I K locus.
